(19) United States
(12) Patent Application Publication
(10) Pub. No.: US 2006/0153823 A1
(43) Pub. Date: Jul. 13, 2006

(54) Compositions and methods for nutritional supplementation

(75) Inventors: John` A. Giordano, West Orange, NJ (US); Charles J. Balzer, Lavalette, NJ (US)

Correspondence Address: PRESTON GATES ELLIS & ROUVELAS MEEDS LLP, 1735 NEW YORK AVENUE, NW, SUITE 500, WASHINGTON, DC, 20006, US

(73) Assignee: Everett Laboratories, Inc.

(21) Appl. No.: 11/034,102

(22) Filed: Jan. 13, 2005

(51) Int. Cl. A61K 38/43; A61K 31/525; A61K 31/714; A61K 31/4415

(52) U.S. Cl. 424/94.1; 514/52; 514/251; 514/350

(57) ABSTRACT

The present invention relates to compositions, that may be swallowable, chewable or dissolvable, comprising various vitamins, and in a specific embodiment, comprise vitamin B₆, vitamin B₁₂, folic acid, and CoQ₁₀, and methods for using these compositions for nutritional supplementation in order to prevent, treat and/or alleviate the occurrence or negative effects of cardiovascular disease.

### FIELD OF THE INVENTION

The present invention relates to compositions comprising various vitamins and methods for using these compositions for nutritional supplementation and in order to prevent, treat and/or alleviate the occurrence or negative effects of cardiovascular disease including congestive heart failure, ischemic heart disease, cardiomyopathy, and hypertension.

### BACKGROUND OF THE INVENTION

Cardiovascular disease continues to be the number one cause of death for both men and women in the United States. Statistics Related to Heart Disease, available at www.health.uab.edu/show.asp?durki=39661 (last visited 1 Dec. 2004). Correlated risk factors for the incidence of cardiovascular disease include blood level elevations of both cholesterol and homocysteine. Id. Recent developments in nutritional research suggest that nutritional supplementation with specific vitamins and minerals, as an adjunct to proper diet, exercise and medical care, can aid in preventing, treating and/or alleviating the occurrence or negative effects of this disease.

Nutrition plays a critical role in maintaining good health, and nutritional supplementation serves a vital role in protecting against poor nutrition and disease. For example, recent research has shown that vitamins and minerals help to prevent, treat and/or alleviate the occurrence or negative effects of cardiovascular disease. While supplementation with certain vitamins and minerals protects against the onset of these diseases, other vitamins and minerals have been found to inhibit the beneficial effects of these certain vitamins and minerals. Specifically, vitamin B₆, vitamin B₁₂, folic acid, and coenzyme Q₁₀ (“CoQ₁₀”) play integral roles in physiological mechanisms that serve to prevent, treat and/or alleviate the occurrence or negative effects of cardiovascular disease. Supplementation with other vitamins and minerals may inhibit the beneficial effects of vitamin B₆, vitamin B₁₂, folic acid, and CoQ₁₀. Thus, when creating or choosing a nutritional supplement, it is essential to understand the physiological needs and risks of individual patients and population groups and the interactions between various vitamins and minerals.

### SUMMARY OF THE INVENTION

The present invention provides compositions and methods of using the compositions for both prophylactic and therapeutic nutritional supplementation. Specifically, the present invention includes vitamins and minerals that prevent, treat and/or alleviate the occurrence or negative effects of cardiovascular disease. The present invention also may be formulated to exclude vitamins and minerals known to inhibit the beneficial effects of the included vitamins and minerals.

In one embodiment, the compositions may include B₆, B₁₂, folic acid, and CoQ₁₀. In another embodiment, the compositions of the present invention may include folic acid in the form of vitamin B₉, folacin, metafolin, folate or natural isomers thereof including (6S)-tetrahydrofolic acid, 5-methyl-(6S)-tetrahydrofolic acid, 5-formyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid or polyglutamyl derivatives thereof.

In another embodiment, the compositions of the present invention may be in a swallowable form. In another embodiment of the present invention, the compositions may be substantially free of one or more of added vitamins, added minerals, and added coenzymes. Yet in another embodiment, the compositions may comprise pharmaceutically acceptable carriers, such as one or more of binders, diluents, lubricants, glidants, colorants, emulsifiers, disintegrants, starches, water, oils, alcohols, preservatives and sugars.

In another embodiment, the compositions of the present invention may comprise about 15 mg to about 45 mg vitamin B₆; about 250 μg to about 750 μg vitamin B₁₂; about 1.25 mg to about 3.75 mg folic acid; and about 50 mg to about 150 mg CoQ₁₀.

Yet in another embodiment, the compositions of the present invention may comprise about 24 mg to about 36 mg vitamin B₆; about 400 μg to about 600 μg vitamin B₁₂; about 2 mg to about 3 mg folic acid; and about 80 mg to about 120 mg CoQ₁₀.

In another embodiment, the compositions of the present invention may comprise about 27 mg to about 33 mg vitamin B₆; about 350 μg to about 550 μg vitamin B₁₂; about 2.25 mg to about 2.75 mg folic acid; and about 90 mg to about 110 mg CoQ₁₀.

In a specific embodiment, the compositions of the present invention may comprise about 30 mg vitamin B₆; about 500 μg vitamin B₁₂; about 2.5 mg folic acid; and about 100 mg CoQ₁₀.

In one embodiment of the present invention, the compositions may be administered to a patient to prevent, treat and/or alleviate the occurrence or negative effects of cardiovascular disease including congestive heart failure, ischemic heart disease, cardiomyopathy, and hypertension.

The present invention also includes methods of administering the compositions of the invention to patients to prevent, treat and/or alleviate the occurrence or negative effects of cardiovascular disease including congestive heart failure, ischemic heart disease, cardiomyopathy, and hypertension.

In one embodiment of the present invention, the methods may utilize compositions comprising vitamin B₆, vitamin B₁₂, folic acid, and CoQ₁₀. In another embodiment, the methods of the present invention may include folic acid in the form of vitamin B₉, folacin, metafolin, folate or natural isomers thereof including (6S)-tetrahydrofolic acid, 5-methyl-(6S)-tetrahydrofolic acid, 5-formyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid or polyglutamyl derivatives thereof.

In one embodiment of the present invention, the methods may utilize compositions in a swallowable form. In another embodiment, the methods may utilize compositions substantially free of other added vitamins, added minerals, and added coenzymes. In another embodiment, the methods may utilize compositions comprising pharmaceutically acceptable carriers, such as one or more of binders, diluents, lubricants, glidants, colorants, emulsifiers, disintegrants, starches, water, oils, alcohols, preservatives and sugars.

In another embodiment, the methods may utilize compositions comprising about 45 mg vitamin B₆; about 250 μg to about 750 μg vitamin B₁₂; about 1.25 mg to about 3.75 mg folic acid; and about 50 mg to about 150 mg CoQ₁₀.

In another embodiment, the methods may utilize compositions comprising about 24 mg to about 36 mg vitamin B₆; about 400 μg to about 600 μg vitamin B₁₂; about 2 mg to about 3 mg folic acid; and about 80 mg to about 120 mg CoQ₁₀.

In another embodiment, the methods may utilize compositions comprising about 27 mg to about 33 mg vitamin B₆; about 350 μg to about 550 μg vitamin B₁₂; about 2.25 mg to about 2.75 mg folic acid; and about 90 mg to about 110 mg CoQ₁₀.

In another embodiment, the methods may utilize compositions comprising about 30 mg vitamin B₆; about 500 μg vitamin B₁₂; about 2.5 mg folic acid; and about 100 mg CoQ₁₀.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### DETAILED DESCRIPTION OF THE INVENTION

It is understood that the present invention is not limited to the particular methodologies, protocols, fillers, and excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “subject,” as used herein, comprises any and all organisms and includes the term “patient.” “Subject” may refer to a human or any other animal.

The phrase “pharmaceutically acceptable,” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “swallowable form” refers to any compositions that do not readily dissolve when placed in the mouth and may be swallowed whole without any chewing or discomfort. In one embodiment, may have a shape containing no sharp edges and a smooth, uniform and substantially bubble free outer coating.

As stated earlier, cardiovascular disease is the number one cause of death for adults in the United States. Recent developments in nutritional research suggest that nutritional supplementation with certain vitamins and minerals, as an adjunct to proper diet, exercise and medical care, can aid in preventing, treating and/or alleviating the occurrence or negative effects of these diseases.

The compositions and methods of the present invention provide means to optimize good health by utilizing vitamin and mineral combinations that specifically aim to prevent, treat and/or alleviate the occurrence or negative effects of cardiovascular disease. The compositions and methods of the present invention may be administered to or directed to a subject such as a human or any other organism. Each of the added vitamins and minerals that can be included in the present invention, including vitamin B₆, vitamin B₁₂, folic acid, and CoQ₁₀, plays a specific role in preventing, treating and/or alleviating the occurrence or negative effects of cardiovascular disease. In a specific embodiment, vitamins and minerals that inhibit these compounds' beneficial effects may be specifically excluded from the compositions and methods of the present invention. Further, in another specific embodiment, other added vitamins and/or minerals can be excluded.

Vitamin B₆ and vitamin B₁₂ are water-soluble nutrients that generally are not stored in the body. These vitamins play a variety of roles within the body. They are included in the compositions and methods of the present invention due to their roles in preventing, treating and/or alleviating the occurrence or negative effects of cardiovascular disease.

Vitamin B₆, vitamin B₁₂, and folic acid help prevent, treat and/or alleviate the occurrence or negative effects of cardiovascular disease through their involvement in the metabolism and breakdown of homocysteine. Homocysteine is an intermediary product produced by metabolism of the amino acid methionine. Elevated levels of homocysteine have been correlated to an increased risk of cardiovascular disease. Maxwell, Suppl 1 BASIC RES. CARDIOL. 165-71 (2003). Elevated levels of homocysteine may lead to increased risk of cardiovascular disease due to this metabolite's numerous deleterious effects on the vascular system including impairing endothelial function, inducing thrombosis, and increasing oxidant stress. Schlaich, 153(2) ATHEROSCLER. 383-89 (2000); Hanratty, 85(3) HEART 326-30 (2001). Efficient breakdown of this intermediary product is necessary to avoid these deleterious effects on the vascular system, and the metabolic breakdown pathways of homocysteine require vitamins B₆, B₁₂ and folic acid. Thus, optimizing the levels of these vitamins has cardio-protective effects by promoting the efficient degradation of homocysteine. Haynes, 16(5) CARDIOVASC. DRUGS THER. 391-9 (2002).

Folic acid is essential for the formation of red and white blood cells within bone marrow and also plays a role in heme formation. RECOMMENDED DIETARY ALLOWANCES, National Research Council, 10^{th} ed., 10 (1989) Folic acid in its active form, tetrahydrofolate, is a coenzyme that is involved in the transfer of methyl groups and it plays a role in DNA synthesis, purine synthesis, and amino acid synthesis, such as the conversion of glycine to serine and the transformation of homocysteine to methionine. The activation of folic acid requires a vitamin B₁₂-dependent transmethylation and vitamin B₁₂ is also necessary for folic acid delivery to tissues. Id.

Folic acid also may have additional physiological effects beyond its role in homocysteine breakdown that protect against cardiovascular disease. Bailey, 133(6) J. NUTR.

S-68S (2003); Doshi, 41 (11) CLIN. CHEM. LAB. MED. 1505-12 (2003); Haynes, supra. For example, folic acid improves the levels and functioning of the health promoting, endothelial-derived compound nitric oxide (NO). Das, 19(7-8) NUTR. 686-92 (2003). Folic acid creates this effect by enhancing the activity of the enzyme nitric oxide synthase, stimulating endogenous tetrahydrobiopterin, and inhibiting generation of intracellular superoxide. All of these actions enhance the half-life of NO thus creating cardioprotective effects. Lucock, 71 MOL. GENET. METAB. 121-38 (2000).

In one embodiment, the compositions and methods of the present invention may include vitamin B₆ in amounts ranging from about 15 mg to about 45 mg. In another embodiment, the compositions and methods of the present invention may include vitamin B₆ in amounts ranging from about 24 mg to about 36 mg. In yet another embodiment, the compositions and methods of the present invention may include vitamin B₆ in amounts ranging from about 27 mg to about 33 mg. In a specific embodiment, the compositions and methods of the present invention may include vitamin B₆ in an amount of about 30 mg.

Vitamin B₁₂ also may be included in the compositions and methods of the present invention. In one embodiment of the present invention, vitamin B₁₂ may be included in amounts ranging from about 250 μg to about 750 μg. In another embodiment, vitamin B₁₂ may be included in amounts ranging from about 400 μg to about 600 μg. In yet another embodiment, vitamin B₁₂ may be included in amounts ranging from about 450 μg to about 550 μg. In a specific embodiment of the present invention, vitamin B₁₂ may be included in an amount of about 500 μg.

Folic acid also may be included in the compositions and methods of the present invention. In one embodiment of the present invention, folic acid may be included in amounts ranging from about 1.25 mg to about 3.75 mg. In another embodiment of the present invention folic acid may be included in amounts ranging from about 2 mg to about 3 mg. In yet another embodiment, folic acid may be included in amounts ranging from about 2.25 mg to about 2.75 mg. In a specific embodiment of the present invention, folic acid may be included in an amount of about 2.5 mg.

CoQ₁₀ belongs to the ubiquinone family of substances. CoQ₁₀ is a fat soluble, vitamin-like substance that is an essential cofactor in the mitochondrial electron transport chain and thus is vital in the production of Adenosine-Tri-Phosphate (ATP). CoQ₁₀ is biosynthesized in the body and shares a common synthetic pathway with cholesterol. Sarter, 16(4) J CARDIOVASC. NURS. 9-20 (2002).

Early studies have documented a deficiency of CoQ₁₀ in patients with a variety of cardiovascular disorders including, but not limited to, congestive heart failure, ischemic heart disease, cardiomyopathy, and hypertension. Id. Studies have indicated that administration of CoQ₁₀ provides a therapeutic benefit to patients with cardiovascular disease. CoQ₁₀ has been shown to significantly reduce blood pressure in patients with hypertension. Id. Administration of CoQ₁₀ to patients with congestive heart failure has been shown to improve myocardial metabolic function, reduce blood pressure, and reduce myocardial thickness. Id. Further, CoQ₁₀ enhances both the magnitude and rate of clinical improvement in patients with ischemic heart disease. Langsjoen et al., 9(2-4) BIOFACTORS. 273-284 (1999). Additionally, studies have suggested CoQ₁₀ may be a benefit in treating patients with cardiomyopathy. Patients treated with CoQ₁₀ showed significant improvement in cardiac function including ejection fraction and cardiac output. J CARDIOVASC. NURS. at 9.

Nutritional supplementation with CoQ₁₀ can help prevent, treat and/or alleviate the occurrence or negative effects of cardiovascular disease. In one embodiment, the compositions and methods of the present invention may include CoQ₁₀ in amounts ranging from about 50 mg to about 150 mg. In another embodiment, the compositions and methods of the present invention may include CoQ₁₀ in amounts ranging from about 80 mg to about 120 mg. In yet another embodiment, the compositions and methods of the present invention may include CoQ₁₀ in amounts ranging from about 90 mg to about 110 mg. In a specific embodiment, the compositions and methods of the present invention may include CoQ₁₀ in an amount of about 100 mg.

The active ingredients are available from numerous commercial sources, and in several active forms or salts thereof, known to those of ordinary skill in the art. Hence, the compositions and methods of the present invention are not limited to comprising or using any particular form of the vitamin or mineral ingredient described herein.

Nutrition is a constantly evolving health science. Nearly as proliferative as research findings correlating nutrients and disease prevention are findings demonstrating that supplementation with some nutrients can be counter-productive to the health needs of specific populations. In a specific embodiment, the compositions and methods of the present invention may be substantially free of other added vitamins, minerals, and coenzymes.

The compositions of the present invention are preferably administered in amounts to patients that provide the supplementation required to alleviate the vitamin deficiencies associated with cardiovascular disease. A preferred dosage of the compositions of the present invention may consist of one or more caplets for human oral consumption. If more than one caplet is used, each individual caplet may be identical to the other caplets, or each may contain only some of the ingredients of the composition, so that the combination of the different caplets comprises a composition of the present invention.

A specific embodiment of the present invention may comprise swallowable compositions. Swallowable compositions are well known in the art and are those that do not readily dissolve when placed in the mouth and may be swallowed whole without any chewing or discomfort. In a specific embodiment of the present invention, the swallowable compositions may have a shape containing no sharp edges and a smooth, uniform and substantially bubble free outer coating.

To prepare the swallowable compositions of the present invention, each of the active ingredients may be combined in intimate admixture with a suitable carrier according to conventional compounding techniques. In a specific embodiment of swallowable compositions of the present invention, the surface of the compositions may be coated with a polymeric film. Such a film coating has several beneficial effects. First, it reduces the adhesion of the compositions to the inner surface of the mouth, thereby increasing the patient's ability to swallow the compositions. Second, the film may aid in masking the unpleasant taste of certain drugs. Third, the film coating may protect the compositions of the present invention from atmospheric degradation. Polymeric films that may be used in preparing the swallowable compositions of the present invention include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia. Pharmaceutical carriers and formulations for swallowable compounds are well known to those of ordinary skill in the art. See generally, e.g., WADE & WALLER, HANDBOOK OF PHARMACEUTICAL EXCIPIENTS (2^{nd} ed. 1994).

Disintegrants also may be included in the compositions of the present invention in order to facilitate dissolution. Disintegrants, including permeabilising and wicking agents, are capable of drawing water or saliva up into the compositions which promotes dissolution from the inside as well as the outside of the compositions. Such disintegrants, permeabilising and/or wicking agents that may be used in the present invention include by way of example and without limitation, starches such as corn starch, potato starch, pre-gelatinized and modified starches thereof, cellulosic agents such as Ac-di-sol, montmorrilonite clays, cross-linked PVP, sweeteners, bentonite, microcrystalline cellulose, croscarmellose sodium, alginates, sodium starch glycolate, gums such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents such as hydroxymethylcellulose, hydroxypropylcellulose and hydroxyopropylmethylcellulose.

Finally, dissolution of the compositions may be facilitated by including relatively small particles sizes of the ingredients used.

In addition to those described above, any appropriate fillers and excipients may be utilized in preparing the swallowable compositions of the present invention so long as they are consistent with the objectives described herein. For example, binders are substances used to cause adhesion of powder particles in granulations. Such compounds appropriate for use in the present invention include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium, and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol, and others known to those of ordinary skill in the art.

Diluents also may be included in the compositions of the present invention in order to enhance the granulation of the compositions. Diluents can include, by way of example and without limitation, microcrystalline cellulose, sucrose, dicalcium phosphate, starches, and polyols of less than 13 carbon atoms, such as mannitol, xylitol, sorbitol, maltitol, and pharmaceutically acceptable amino acids, such as glycin, and their mixtures.

Lubricants are substances used in composition formulations that reduce friction during composition compression. Lubricants that may be used in the present invention include, by way of example and without limitation, stearic acid, calcium stearate, magnesium stearate, zinc stearate, talc, mineral and vegetable oils, benzoic acid, poly(ethylene glycol), glyceryl behenate, stearyl fumarate, and others known to those of ordinary skill in the art.

Glidants improve the flow of powder blends during manufacturing and minimize composition weight variation. Glidants that may be used in the present invention include by way of example and without limitation, silicon dioxide, colloidal or fumed silica, magnesium stearate, calcium stearate, stearic acid, cornstarch, talc and others known to those of ordinary skill in the art.

Colorants also may be included in the nutritional supplement compositions of the present invention. As used herein, the term “colorant” includes compounds used to impart color to pharmaceutical preparations. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and others known to those of ordinary skill in the art. Coloring agents also can include pigments, dyes, tints, titanium dioxide, natural coloring agents such as grape skin extract, beet red powder, beta carotene, annato, carmine, turmeric, paprika, and others known to those of ordinary skill in the art. It is recognized that no colorant is required in the nutritional supplement compositions described herein.

If desired, compositions may be sugar coated or enteric coated by standard techniques. The unit dose forms may be individually wrapped, packaged as multiple units on paper strips or in vials of any size, without limitation. The swallowable, chewable or dissolvable compositions of the invention may be packaged in unit dose, rolls, bulk bottles, blister packs and combinations thereof, without limitation.

The swallowable compositions of the present invention may be prepared using conventional methods and materials known in the pharmaceutical art. For example, U.S. Pat. Nos. 5,215,754 and 4,374,082 relate to methods for preparing swallowable compositions. Further, all pharmaceutical carriers and formulations described herein are well known to those of ordinary skill in the art, and determination of workable proportions in any particular instance will generally be within the capability of the person skilled in the art. Details concerning any of the excipients of the invention may be found in WADE & WALLER, supra. All active ingredients, fillers and excipients are commercially available from companies such as Aldrich Chemical Co., FMC Corp, Bayer, BASF, Alexi Fres, Witco, Mallinckrodt, Rhodia, ISP, and others.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

### Example 1

A composition of the following formulation was prepared in caplet form by standard methods known to those skilled in the art:

| | | |
|---|---|---|
| Vitamin B₆ | 30 | mg |
| Vitamin B₁₂ | 500 | μg |
| Folic acid | 2.5 | mg |
| CoQ₁₀ | 100 | mg |

### Example 2

A study is undertaken to evaluate the effectiveness of the compositions of the present invention in the treatment of patients. The objective of the study is to determine whether oral intake of the compositions results in an improvement of the nutritional status with regard to specific vitamins and minerals contained in the administered compositions.

A double-blind, placebo controlled study is conducted over a six-month period. A total of 120 subjects, aged 30-45 years, are chosen for the study. An initial assessment of the nutritional status of each subject is conducted. Vitamin B₆ is measured by a radioenzymatic assay method wherein serum is incubated with apoenzyme tyrosine-decarboxylase, C₁₄ labeled tyrosine is added to start the enzymatic reaction which is stopped with HCl. Subsequently the free C₁₄-labelled CO₂ is adsorbed by a KOH impregnated filtering paper. The measured C₁₄ activity is directly proportional to the B₆ concentration. Vitamins B₁₂ and folic acid are measured by quantitative radioassay methods using purified intrinsic factor and purified folate binding protein. CoQ₁₀ is measured by using column-switching high performance liquid chromatography (HPLC). Determination of CoQ₁₀ is performed on a reversed-phase analytical column with ultraviolet detection at 275 nm. The mobile phase contains 10% (v/v) isopropanol in methanol at a flow rate of 1.5 m./min. This method allows for the detection of 0.1 microg/ml CoQ₁₀ in plasma (S/N=3).

The 120 subjects are separated into 4 separate groups of 30 subjects. In a first group comprising men, and in a second group comprising women, each subject is administered one dosage form of the composition as described in Example 1 once a day. In a third group comprising men and a fourth group comprising women, each subject is administered one placebo dosage form once a day. Thus, dosage form administration occurs every 24 hours. No other nutritional supplements are taken by the subjects during the assessment period.

An assessment of the nutritional status of each subject is conducted utilizing methods described above at one month intervals for a six month period. The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 1, 2, 3, 4, 5 and 6 months, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant improvement in the nutritional status of all vitamin and mineral levels measured is observed in the treated subjects over the controls upon completion of the study. Therefore, the study confirms that oral administration of the compositions of the present invention is effective in improving the nutritional status of patients.

While specific embodiments of the present invention have been described, other and further modifications and changes may be made without departing from the spirit of the invention. All further and other modifications and changes are included that come within the scope of the invention as set forth in the claims. The disclosure of all publications cited above are expressly incorporated by reference in their entireties to the same extent as if each were incorporated by reference individually.

What is claimed is:

1. A composition comprising vitamin B₆, vitamin B₁₂, folic acid, and CoQ₁₀.

2. The composition of claim 1, wherein said folic acid is selected from the group consisting of vitamin B₉, folacin, metafolin, folate, (6S)-tetrahydrofolic acid or a polyglutamyl derivative thereof, 5-methyl-(6S)-tetrahydrofolic acid or a polyglutamyl derivative thereof, 5-formyl-(6S)-tetrahydrofolic acid or a polyglutamyl derivative thereof, 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof.

3. The composition of claim 1, wherein said composition is substantially free of one or more of other added vitamins, added minerals, and added coenzymes.

4. The composition of claim 1, further comprising pharmaceutically acceptable carriers.

5. The composition of claim 4, wherein said pharmaceutically acceptable carriers are selected from one or more of the group consisting of binders, diluents, lubricants, glidants, colorants, emulsifiers, disintegrants, starches, water, oils, alcohols, preservatives and sugars.

6. The composition of claim 1, wherein said vitamin B₆ is present in the range of about 15 mg to about 45 mg.

7. The composition of claim 1, wherein said vitamin B₁₂ is present in the range of about 250 μg to about 750 μg.

8. The composition of claim 1, wherein said folic acid is present in the range of about 1.25 mg to about 3.75 mg.

9. The composition of claim 1, wherein said CoQ₁₀ is present in the range of about 50 mg to about 150 mg.

10. The composition of claim 1, wherein said composition comprises about 15 mg to about 45 mg vitamin B₆; about 250 μg to about 750 μg vitamin B₁₂; about 1.25 mg to about 3.75 mg folic acid; and about 50 mg to about 150 mg CoQ₁₀.

11. The composition of claim 1, wherein said composition comprises about 24 mg to about 36 mg vitamin B₆; about 400 μg to about 600 μg vitamin B₁₂; about 2 mg to about 3 mg folic acid; and about 80 mg to about 120 mg CoQ₁₀.

12. The composition of claim 1, wherein said composition comprises about 27 mg to about 33 mg vitamin B₆; about 450 μg to about 550 μg vitamin B₁₂; about 2.25 mg to about 2.75 mg folic acid; and about 90 mg to about 110 mg CoQ₁₀.

13. The composition of claim 12, wherein said vitamin B₆ is present in the amount of about 30 mg.

14. The composition of claim 12, wherein said vitamin B₁₂ is present in the amount of about 500 μg.

15. The composition of claim 12, wherein said Folic Acid is present in the amount of about 2.5 mg.

16. The composition of claim 12, wherein said CoQ₁₀ is present in the amount of about 100 mg.

17. The composition of claim 1, wherein said vitamin B₆ is present in the amount of about 30 mg; said vitamin B₁₂ is present in the amount of about 500 μg; said folic acid is present in the amount of about 2.5 mg; and said CoQ₁₀ is present in the amount of about 100 mg.

18. The composition of claim 1, wherein said composition is administered to a patient.

19. The composition of claim 18, wherein said composition is administered to said patient orally.

20. The composition of claim 18, wherein said composition is administered to said patient to prevent, treat and/or alleviate the occurrence or negative effects of cardiovascular disease.

21. The composition of claim 20, wherein said cardiovascular disease is selected from one or more of the group consisting of congestive heart failure, ischemic heart disease, cardiomyopathy, and hypertension.

22. A method comprising administering a composition comprising vitamin B₆, vitamin B₁₂, Folic Acid, and CoQ₁₀.

23. The method of claim 22, wherein said folic acid is selected from the group consisting of B₉, folacin, metafolin, folate, (6S)-tetrahydrofolic acid or a polyglutamyl derivative thereof, 5-methyl-(6S)-tetrahydrofolic acid or a polyglutamyl derivative thereof, 5-formyl-(6S)-tetrahydrofolic acid or a polyglutamyl derivative thereof, 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof.

24. The method of claim 22, wherein said composition is substantially free of one or more of other added vitamins, added minerals, and added coenzymes.

25. The method of claim 22, further comprising pharmaceutically acceptable carriers.

26. The method of claim 25, wherein said pharmaceutically acceptable carriers are selected from one or more of the group consisting of binders, diluents, lubricants, glidants, colorants, emulsifiers, disintegrants, starches, water, oils, alcohols, preservatives and sugars.

27. The method of claim 22, wherein said vitamin B₆ is present in the range of about 15 mg to about 45 mg.

28. The method of claim 22, wherein said vitamin B₁₂ is present in the range of about 250 μg to about 750 μg.

29. The method of claim 22, wherein said Folic Acid is present in the range of about 1.25 mg to about 3.75 mg.

30. The method of claim 22, wherein said CoQ₁₀ is present in the range of about 50 mg to about 150 mg.

31. The method of claim 22, wherein said composition comprises about 15 mg to about 45 mg vitamin B₆; about 250 μg to about 750 μg vitamin B₁₂; about 1.25 mg to about 3.75 mg folic acid; and about 50 mg to about 150 mg CoQ₁₀.

32. The method of claim 22, wherein said composition comprises about 24 mg to about 36 mg vitamin B₆; about 400 μg to about 600 μg vitamin B₁₂; about 2 mg to about 3 mg folic acid; and about 80 mg to about 120 mg CoQ₁₀.

33. The method of claim 22, wherein said composition comprises about 27 mg to about 33 mg vitamin B₆; about 450 μg to about 550 μg vitamin B₁₂; about 2.25 mg to about 2.75 mg folic acid; and about 90 mg to about 110 mg CoQ₁₀.

34. The method of claim 33, wherein said vitamin B₆ is present in the amount of about 30 mg.

35. The method of claim 33, wherein said vitamin B₁₂ is present in the amount of about 500 μg.

36. The method of claim 33, wherein said Folic Acid is present in the amount of about 2.5 mg.

37. The method of claim 33, wherein said CoQ₁₀ is present in the amount of about 100 mg.

38. The method of claim 22, wherein said vitamin B₆ is present in the amount of about 30 mg; said vitamin B₁₂ is present in the amount of about 500 μg; said folic acid is present in the amount of about 2.5 mg; and said CoQ₁₀ is present in the amount of about 100 mg.

39. The method of claim 22, wherein said composition is administered to a patient.

40. The method of claim 39, wherein said composition is administered to said patient orally.

41. The method of claim 39, wherein said composition is administered to said patient to prevent, treat and/or alleviate the occurrence or negative effects of cardiovascular disease.

42. The method of claim 41, wherein said cardiovascular disease is selected from one or more of the group consisting of congestive heart failure, ischemic heart disease, cardiomyopathy, and hypertension.
